(19) United States
(12) Patent Application Publication
(10) Pub. No.: US 2001/0032340 A1
(43) Pub. Date: Oct. 18, 2001

(54) Controlling offspring's sex ratio by targeting transgenes onto the sex chromosomes

(75) Inventors: Chengyu Liu, Boyds, MD (US); Franklin Costantini, Pelham, NY (US); Jin Wang, Verona, NJ (US)

Correspondence Address: Chris A. Caseiro, Pierce Atwood, One Monument Square, Portland, ME, 04101, US

(73) Assignee: Chengyu Liu

(21) Appl. No.: 09/749,709

(22) Filed: Dec. 27, 2000

Related U.S. Application Data
(60) Provisional application No. 60/173,096, filed on Dec. 27, 1999.

(51) Int. Cl. A01K 67/027

(52) U.S. Cl. 800/18; 800/21

(57) ABSTRACT

A method for controlling the sex ratio of offspring by targeting transgenes. The method involves: (a) selecting or creating a transgene whose expression can interfere with sperm's ability to undergo fertilization, and whose gene products (mRNA and protein) do not diffuse freely among inter-connected spermatids; (b) placing the transgene under the regulatory control of post-meiotic spermatogenesis-specific promoter; and (c) using the transgene to generate transgenic animals in the way that the transgene is inserted onto one of the two sex chromosomes.

### CROSS-REFERENCE TO RELATED APPLICATION

This application claims the priority benefit of U.S. provisional application Ser. No. 60/173,096, filed Dec. 27, 1999, of the same title and by the same inventors.

### BACKGROUND OF THE INVENTION

1. Field of the Invention

The present invention relates to methods and compositions of matter useful for making transgenic animals whose offspring's sex ratio can be altered through the expression of said transgene integrated onto one of the two sex chromosomes.

2. Description of the Prior Art

The majority of animal species consists of two sexes, male and female. For many species, males and females are different in many aspects such as body size, growth rate and behavior. Certain features are even unique to one sex. In farm animals, one sex may be advantageous over the other for producing desired products such as meat, milk, egg, and wool. Thus, controlling the birth ratio of the two sexes of livestock is economically important, since it allows farmers to take full advantage of the differences between the two sexes.

In mammals, as well as in many other organisms, sex is determined by the sex chromosome (X or Y) of the sperm. Normally, an egg fertilized by a sperm that contains the Y chromosome (Y-sperm) will develop into a male, and on the other hand, an egg fertilized by a sperm that contains the X chromosome (X-sperm) will become a female. This genetic mechanism determines that approximately equal numbers of males and females are born. The key for controlling sex is to select the right sperm (X or Y) for fertilization. During the past three decades, several immunological, mechanical, and chemical methods have been tested to separate the X and Y sperm from collected semen. The idea is to use separated sperm to fertilize eggs by artificial insemination or in vitro fertilization with the hope to change the sex ratio of the offspring. Although dozens of patents have been issued to cover these techniques, these methods are generally considered inefficient and unreliable. Further, they require equipment and technical expertise that are not available to most farmers. Recently, Sry (Sex-determine Region Y) gene has been identified as an important male determinant in mammals. Several laboratories have tried to preferentially increase the percentage of male newborns by expressing Sry gene in transgenic animals. However, only 30% of the XX transgenic mice (normally female) grew male organs such as testis, and these sex-reversed animals were sterile.

Therefore, what is needed is a novel approach to entirely eliminate the production of either X or Y sperm in the testis so that all the offspring of such male will be the same sex, either male or female. What is also needed is such an approach,such that the animals are genetically modified so that this special trait can be passed from one generation to another. Further, what is needed is such an approach that is distinct from trying to separate the X and Y sperm in the semen. Such an approach should control sex ratio more precisely, and also eliminate the tricky separation process for each semen sample.

### SUMMARY OF THE INVENTION

Here, we developed a novel approach that could entirely eliminate the production of either X or Y sperm in the testis. Therefore, all the offspring of such male will be the same sex, either male or female. The animals are genetically modified so that this special trait can be passed from one generation to another. This approach is clearly better than those trying to separate the X and Y sperm in the semen. It can control sex ratio more precisely, and also eliminate the tricky separation process for each semen sample.

The method of the present invention involves: (a) selecting or creating a transgene whose expression can interfere with sperm's ability to undergo fertilization, and whose gene products (mRNA and protein) do not diffuse freely among inter-connected spermatids; (b) placing the said transgene under the regulatory control of post-meiotic spermatogenesis-specific promoter; (c) using the said transgene to generate transgenic animals in the way that the transgene is inserted onto one of the two sex chromosomes. Expression of the said transgene can reduce the ability of the sperm with one particular sex chromosome to fertilize eggs and develop into new individuals, and consequently controlling the sex ratio of the offspring. This method can also be modified by replacing the post-meiotic spermatogenesis-specific promoter with a promoter that expresses during embryogenesis (after fertilization), and replacing the transgene that is toxic to sperm with a transgene that is toxic to early embryos. Embryonic expression of a sex chromosome-linked toxin transgene disrupts the normal development of embryos with one particular sex chromosome, and only allows embryos with preferred sex to develop into viable individuals. This modified method can be used not only in organisms using X and Y chromosomes but also Z and W chromosomes for sex determination. This method allows farmers to choose the preferred sex of farm animals to produce dairy and meat products and allows laboratory animal suppliers to specifically increase the birth ratio of the preferred gender.

These and other advantages of the present invention will become apparent upon review of the following description, the accompanying drawings, and the appended claims.

### BRIEF DESCRIPTION OF THE DRAWING

The FIGURE is a simplified representation of a method of the present invention for altering the sex chromosomes in animals.

### DETAILED DESCRIPTION OF THE INVENTION

### Methods

1. Rationale and Strategy

It has been demonstrated in transgenic mice that a tissue or a cell type can be specifically eliminated by expressing a toxic gene product. Therefore, post-meiotic expression of a toxin gene in the haploid spermatids destroys the sperm that contain the toxin transgene. When the transgene is integrated into one of the two sex chromosomes (X or Y), only the spermatids containing that particular sex chromosome are disabled while the spermatids containing the other sex chromosome may function normally. Such male transgenic animals can only produce one type of sperm, and hence all of their offspring should be the same sex. A major challenge to the above theory is the fact that the haploid spermatids remain connected by cytoplasmic bridges (˜1 um), i.e. they are syncytial. Therefore, the toxin could diffuse into the spermatids that do not contain the transgene, leading to the destruction of all sperm rather than only the half with the toxin transgene (Braun et. al., 1989, see review Davies and Willison, 1993). However, it is possible to solve this problem by choosing a toxin gene whose products can not diffuse freely among the inter-connected spermatids, or by creating transgenes with limited diffusion characteristics by combining cellular localization DNA sequences to the toxin transgenes. A technique for achieving that is represented in a simplified manner in the accompanying FIGURE.

In the current invention, a herpes simplex type-1virus thymidine kinase (HSV-tk) gene is used as the toxin transgenes. It has been previously shown that HSV-tk contains a spermatogenesis-specific cryptic promoter and its expression disrupt spermatogenesis (Palmiter et al. 1984; Ellison and Bishop, 1988; Braun et al., 1990; Wilkie et al., 1991; Al-Shawi et al., 1991; Huttner et al., 1993; Bernier et al., 1994; Salomon et al., 1995; Ellison et al., 1995; Ellison and Bishop, 1996; Al-Shawi et al., 1998). One common phenotype for all these HSV-tk transgeneic mice is that the male mice can not transmit the HSV-tk transgene to the next generation although many of these males are fertile. Gondo et al. (1994) It has been reported that embryonic stem (ES) cell lines containing the HSV-tk transgene could not transmit through the male germ-line either. All these results together with the report that HSV-tk protein was preferentially distributed at the perinuclear region of transfected cell (Haarr and Flatmark, 1987) implies that the diffusion of HSV-tk gene products (mRNA and protein) among the inter-connected spermatids is limited. Therefore, HSV-tk is an ideal candidate for use in the sex control project. Of course, it is possible to find other genes with similar properties. Alternatively, such toxin genes can be created by linking cellular localization DNA sequences to a variety of genes that may interfere with sperm's function, such as genes that cause cell death, regulatory genes that block sperm development and maturation, mutated cytoskeleton or energy-producing genes involved in sperm motility, and genes involved in gamete recognition, penetration and fusion.

The toxic transgene can be inserted onto the sex chromosome by a variety of methods, and several examples are briefly described below.

(1) Gene-targeting using embryonic stem (ES) cells technology

(a) Targeting the transgene onto the sex chromosome of ES cells using the standard homologous recombination method.

(b) Microinjecting the positive ES cells into recipient embryos to make chimeric mice.

(c) Breeding the chimeric mice to pass the ES-derived germ cells to the next generation to obtain the desired transgenic animals.

(2) Gene-targeting using animal cloning technology

(a) Targeting the transgene onto the sex chromosome of suitable nuclear donor cells (such as fibroblasts derived from embryos) by using the standard homologous recombination method.

(b) Transferring the nuclei of the donor cells into enucleated oocytes by cell fusion or nuclear transfer.

(c) Activating the reconstituted oocytes and transfering them into pseudo pregnant foster mothers to allow the embryos to develop into individuals with the transgene inserted onto the desired chromosome.

(3) Random integration using ES cell or cloning technology

(a) Co-transfecting the HSV-tk transgene with a selection marker gene (such as neomycin-resistant gene) into ES or nuclear donor cells by various transfection methods (such as electroporation).

(b) Picking and growing neomycin-resistant clones, and examining the transgene integration site by florescence in situ hybridization (FISH). Expanding the clones with the transgene inserted onto the desired sex chromosome.

(c) Injecting the ES cells into embryos to make chimeric animals, or cloning the animals using the donor cells' nuclei.

(4) Random insertion of the transgene into gametes or germ cells

Using a variety of methods such as pronuclear injection, retroviral vector transfection, lipofection, and sperm incubation to randomly insert the transgene into the genome. Then, using FISH to screen the transgenic founders to identify individuals with the transgene inserted onto the desired sex chromosome.

Methods (3) and (4) use a random integration mechanism. The advantage for these methods is they are relatively easy to use to make the DNA constructs. The disadvantage is it is necessary to screen a considerable number of cell clones or individuals in order to find the one with the transgene inserted onto the desired sex chromosome. Methods (1) and (2) use a homologous recombination method to specifically target the transgene onto the desired sex chromosome, and therefore eliminate the need for screening using FISH. However, it involves more to make the gene-targeting DNA constructs. In addition to the selectable marker genes that need to be incorporated into the targeting construct, two flanking homologous DNA fragments are also necessary for the homologous recombination events to occur. As more and more DNA sequence data are available from the sex chromosomes, many loci can be used as target sites for inserting the transgene. The general rules are that the target loci should be accessible by the transcription machinery for transcription in spermatids and the insertion of the transgene into those sites do not cause abnormal phenotype to the transgenic animals.

Since the transgenic males can not transmit the HSV-tk transgene from one generation to the next, an inducible (such as the tetracyclin inducible system) or conditional system (such as Cre/IoxP) need to be used. For example, IoxP sites flanked by intervening DNA sequence can be inserted between the promoter and the transgene to prevent the expression of the HSV-tk transgene. Therefore, the transgene can be transmitted through generations to maintain the lines. When needed for sex control, the HSV-tk transgene can be activated by removing the intervening DNA sequences (between the two IoxP sites) through mating with females containing the Cre recombinase gene. The Cre transgenic animals can be purchased through commercial sources or can be created using standard transgenic methods. For transgenes targeted on the X chromosome, it is possible to maintain the transgenic line through females (mother to daughter). Therefore, it is not necessary to use the inducible or conditional systems after the line is created.

2. Modified Method

The above technology can be modified by replacing the post-meiotic spermatogenesis-specific promoters with promoters that express during embryogenesis (after fertilization), and repalcing the toxin transgene that disrupts the sperm's function to a transgene that affects embryonic development or viability. When such transgenes are inserted onto one of the two sex chromosomes, the expression of the transgenes during early embryogenesis disrupts the normal development of embryos with one particular sex chromosome, and allows only embryos with preferred sex to develop into viable individuals. This modified method will reduce the litter size by a half. However, if it is combined with superovulation (injecting hormones to increase the number of eggs per ovulation), it is a useful method for sex selection.

This modified method needs an inducible or conditional system for maintaining the transgene through generations since all embryos with the transgene are killed and therefore can not transmit to the next generation. For example, the toxin transgene can be prevented from transcription by inserting a IoxP site-flanked intervening sequence between the promoter and the transcription unit. Such inactive transgene can be passed from generation to generation. When needed for sex control, the transgenic animals can mate with animals containing a Cre recombinase gene controlled by gametogenesis-specific promoters. The Cre recombinase will activate the transgenes in the gametes, the embryos developed from such gametes will be eliminated, and only the embryos with the sex chromosome that does not contain the transgene can develop into individuals.

The modified method can be used not only in organisms using X and Y but also in organisms using Z and W chromosomes to determine sex. In organisms using a ZW system to determine sex, the females are heterogametic (Z eggs and W eggs) while the males are homogametic (all sperm contain Z chromosome). Therefore, transgenic females are used for sex control. For species which lay a large number of eggs during each ovulation (such as many aquatic species, amphibians, insects and plants), elimination of half of the early embryos should not significantly influence its reproduction since a majority of the embryos can not develop into adults anyway. However, for those species whose eggs themselves are valuable commodities (such as birds), it needs to be carefully evaluated if the benefit of being able to control the sex of chicks is more than the value of the half of the eggs being destroyed.

3. Practical Procedures for Proving the Method in Laboratory Mouse

This technology can be used in all organisms that use sex chromosomes for sex determination, but it only needs to be tested in one species to prove the principles. Currently, mouse is the species of choice for proving the method. Below is an example of the specific protocols that can be used to generate the desired transgenic mice.

1). Purchasing the HSV-tk gene, neomycin-resistant gene (neo), diphtheria toxin (DT) from commercial sources, such as Stratagene.

2) Making the IoxP site DNA by annealing two single-strand oligodeoxynucleotides.

3) Cloning of the flanking homologous DNA fragments needed for homologous recombination by polymerase chain reaction (PCR) using 129 mouse geneomic DNA as a template. For examples, the transgene can be targeted onto the Hprt locus (see Melton et al., 1984 for gene sequences) of the X chromosome, or the Tspy pseudogene locus (see Vogel et al., 1998 for sequences) of the Y chromosome. Specifically, a ˜4 Kb fragment can be amplified between exon 6 and exon 7 of the Hprt gene using the primer CAGTACAGCCCCAAAATGGT and primer GAGGTCCTTTTCACCAGCAA; a ˜1.3 Kb fragment can be amplified between exon 8 and exon 9 of the Hprt gene using primer AGTTTGTTGTTGGATATGCC and primer CCTCTTAGATGCTGTTACTG; a ˜1 Kb DNA fragment can be amplified from the 5′ end of the Tspy pseudogene by using primers AGGAGAGTGTGGGCATG and AAATGCACAATCTAAAGC; and a ˜1.5 Kb fragement can be amplified from the 3′ end of the Tspy pseudogene by using primers CTCCAAGGACTGCTCTCA and AAACATGAGAAACATGGTA.

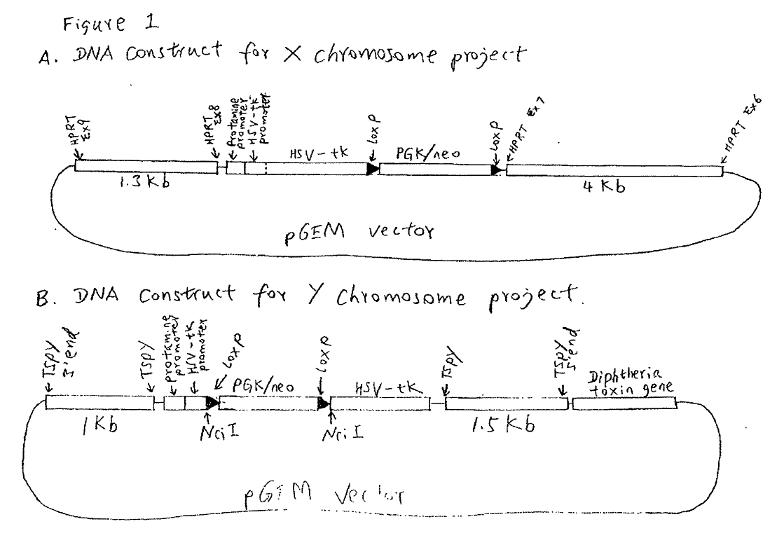

4) Assembling the above DNA fragments according to FIG. 1 to make the gene-targeting constructs for X and Y chromosomes.

5) Electroporating the DNA constructs into ES cells, and plating the ES cells in medium containing G418 to allow neomycin-resistant colonies to appear. Since the Hprt gene itself is a selectable marker, adding 6-thioguanine (6-TG) in addition to G418 into the selection medium will increase the chance of targeted clones versus randomly integrated clones for the X chromosome project. For the Y chromosome project, the diphtheria toxin (DT) gene in the targeting construct serves as a negative selectable marker, which can also increase the chance of targeted-clones versus randomly integrated-clones.

6) Growing the ES cells for 7-10 days in the selection media, and then picking the resistant colonies and expanding them in multi-well culture plates.

7) Duplicating the multi-well plates. Cryopreserving one set of plates to store the ES clones, and preparing genomic DNA from the other set of plates.

8) Analyzing the genomic DNA samples by PCR and Southern blotting to identify homologous recombination events. ES clones with the transgene targeted onto the correct chromosome locus are further propagated and stored as positive clones.

9) Injecting the positive clones into blastocyst stage mouse embryos. Transferring the injected embryos into the uterus of pesudopregnant foster mothers to allow chimeric mice to be born.

10) Breeding the chimeric mice with wild-type mice to allow transmission of the transgene to the next generation. For the X chromosome project, it is important to breed female chimeras with wild type males for germ-line transmission of the transgene since the transgene can not be transmitted through sperm. For the Y chromosome project, it is necessary to breed male chimeras with wild type females to transmit the inactive form of the transgene.

11) Screening the offspring of the above mating by PCR or Southern blotting using tail DNA to identify mice with the transgene.

12) For the X chromosome project, the transgene can be transmitted through generations by females, and the males are used for sex control since all of their offspring are the same sex.

13) For the Y chromosome project, the inactive form of the transgene is maintained through males. When needed for sex control, the transgene can be activated through mating with females containing Cre recombinase activity. The Cre mice can be purchased from commercial laboratory animal suppliers, or can be made by standard transgenic methods.

The following references provide background information related to the present invention. To the extent any reference is specifically cited in the Detailed Description, it is incorporated herein by reference for the purpose relied upon in describing the invention.

Al-Shawi, R., J. Burke, H. Wallace, C. Jones, S. Harrison, D. Buxton, S. Maley, A. Chandley, and J. O. Bishop (1991) Mol. Cell Biol. 11:4207-4216.

Al-Shawi, R., J. Burke, C. T. Jones, J. P. Simons, J. O. Bishop (1988) Mol. Cell. Biol. 8:4821-4828.

Bernier F., S. L. Guerin, M. Ouellet, G. Pelletier, and F. Pothier (1994) Transgenics, 1:225-240.

Braun, R. E., R. R. Behringer, J. J. Peschon, R. L. Brinster, and R. D. Palmiter (1989) Nature, 337:373-376.

Braun, R. E., D. Lo, C. A. Pinkert, G. Widera, R. A. Flaveill, R. D. Palmiter, and R. L. Brinster (1990) Biol. Reprod. 43:684-693.

Capel, B., A. Swain, S. Nicolis, A. Hacker, M. Walter, P. Koopman, P. Goodfellow, and R. Lovell-Badge (1993) Cell, 73:1019-1030.

Davies, P.O. and K. R. Willison (1993) Sem. Dev. Biol., 3:179-188.

Ellison A. R. and J. O. Bishop (1998) Biochim Biophys Acta. 1442:28-38.

Ellison, A. R., H. Wallace, R. Al-Shawi, and J. O. Bishop (1995) Mol. Reprod. Dev., 41:425-434.

Ellison, A. R. and J. O. Bishop (1996) Nucleic Acids Res. 24:2073-2079.

Erickson R. P. (1990) Trens Genet., 6:264-269

Flatmark T. and L. Haarr (1987) J. Gen. Virol., 68:2817-2829.

Gondo, Y., K. Nakamura, K. Nakao, T. Sasaoka, K. Ito, M. Kimura, and M. Katsuki (1994) Biochem. Biophys. Res. Commun. 202:830-837.

Hendriksen, P. J. M., J. W. Hoogerbrugge, A. P. N. Themmen, M. H. M. Koken, J. H. J. Hoeijmakers, B. A. Oostra, T. V. D. Lende, and J. A. grootegoed (1995) Dev. Biol. 170:730-733.

Huttner, K. M., J. Pudney, D. S. Milstone, D. Ladd, and J. G. Seidman (1993) Biol. Reprod. 49:251-261.

Melton, D. W., D. S. Konecki, J. Brennand, and C. T. Caskey (1984) Proc. Natl. Acad. Sci. USA, 81:2147-2151.

Nagamine, C. M., K. Chan, L. E. Hake, and Y-F. C. Lao (1990) Genes Dev., 4:63-74.

Palmiter, R.D., T. M. Wilkie, H. Y. Chen, and R. L. Brinster (1984) Cell, 36:869-877.

Salomon B., S. Maury, L. Loubiere, M. Caruso, R. Onclercq, and D. Klatzman (1995) Mol. Cell Biol., 15:5322-5328.

Shannon, M. and M. A. Handel (1993) Biol. Reprod., 49:770-778.

Vogel, T., H. Boettger-Tong, I. Nanda, F. Dechend, A. I. Agulnik, C. E. Bishop, M. Schmid, and J. Schmidtke (1998) Chromosome Res. 6:35-40.

Wilkie. T. M., R. E. Braun, W. J. Ehrman, R. D. Palmiter, and R. E. Hammer (1991) Genes Dev., 5:38-48.

The principles and features of the present invention, described in examples above, will be understood more broadly from the following claims. The claims are intended to cover the invention as described and all equivalents.

We claim:

1. A method for producing transgenic animals whose somatic/germ cells contain one or more transgenes, wherein expression of the transgenes result in alteration of the sex ratio of the offspring of said animals, the method comprising the steps of:
a. preparing a transgene including in operable association (a) at least one expression regulatory sequence (promoter) functional in a post-meiotic spermatogenesis-specific way; (b) a DNA sequence encoding a toxic gene whose expression can interfere with sperm's ability to undergo fertilization; (c) an optional DNA sequence encoding a selectable marker such as neomycin-, hygromycin- or puromycin-resistance gene, Hprt selection cassette, and diphtheria toxin gene; (d) an optional IoxP site-flanked intervening DNA sequence inserted between the post-meiotic promoter and the toxin gene, and said intervening sequence can prevent the transcription of the toxin transgene unless it is removed by Cre recombinase; (e) an optional cellular localization signal sequence that restricts the ability of the mRNA and protein from the said transgenes to randomly diffuse among the inter-connected haploid spermatids; and (f) two optional flanking DNA sequences allowing said transgene to be inserted onto specific loci of the sex chromosome (the X or Y chromosome) by homologous recombination method;
b. creating transgenic animals using the said transgene so that the transgene is inserted onto one of the two sex chromosomes; and
c. mating the males of the said transgenic animals with animals containing Cre recombinase activity to activate the said transgene and identifying at least one transgenic animal with desirable reproduction feature, specifically, alteration of offspring's sex ratio.

2. The method according to claim 1 wherein said animals include all mammals and non-mammal organisms using X and Y chromosomes to determine sex, and unisexual flower plants.

3. The method according to claim 1, wherein said transgene is selected from the group consisting of Herpes Simplex Virus thymidine kinase gene (HSV-tk), its mutated or truncated genes and any other toxic genes with characters of a) its expression can interfering with sperm's ability to undergo fertilization; and b) its mRNA/protein products act in a no-random diffusion fashion among the interconnected spermatids.

4. The method according to claim 1 wherein the offspring's desirable sex percentage of said transgenic animals is from 50% to 100%.

5. The method according to claim 1 wherein said post-meiotic spermatogenesis-specific promoter is selected from the group consisting of promoters from HSV-TK gene, protamine family genes, kit, angiotensin converting enzyme (Ace) gene, CaM3 gene, TP-1 gene, TP-2 gene, cytochrome cs gene, PSK-C3 gene, H2B gene, Mea gene, delta-actin gene, proacrosin gene, Idh gene, M-alpha-3, 7 tubulin gene, hsp70.1 gene, Wnt.gene and zinc finger Y gene or any promoter which can trigger post-meiotic expression of said transgenes.

6. The method according to claim 1 wherein said the DNA sequence for X-chromosome specific targeting is Hprt locus or other X-linked sequences whose disruption will not cause abnormal phenotype in transgenic animals.

7. The method according to claim 1 wherein said DNA sequence for Y-chromosome specific targeting is Tspy pseudogene or any Y-linked sequences whose disruption will not cause abnormal phenotype in transgenic animals.

8. The method according to claim 1 wherein the post-meiotic spermatogenesis-specific promoter is replaced with an embryonically-expressed promoter, and the transgene that disrupts sperm's function is replaced by a transgene that interferes with embryonic development or viability, the method further comprising the steps of inserting such transgene onto one of the two sex chromosomes prevents embryos with one particular sex chromosome from developing into individuals, preparing a transgene which comprises in operable association of (1) at least one expression regulatory sequence (promoter) which expresses in early stage embryos but not during spermatogenesis (for XY organisms) or oogenesis (for ZW organisms); (2) a DNA sequence encoding a toxic gene (such as diphtheria toxin gene and ricin gene) whose expression can kill the embryo or block the normal development of embryos; (3) a IoxP site-flanked intervening DNA sequences inserted between the promoter and the toxin gene, and the said intervening DNA sequences can prevent the toxin gene transcription unless it is removed by Cre recombinase; (4) an optional DNA sequence encoding a selectable marker such as neomycin-, hygromycin- or puromycin-resistance gene, Hprt selection cassette, and diphtheria toxin gene; (5) two optional flanking DNA fragments allowing said transgene to be inserted onto specific loci of the sex chromosome by homologous recombination method, creating transgenic animals, and breeding of the transgenic animals with animals that contain a Cre recombinase transgene driven by spermatogenesis-(for XY organisms) or oogenesis- (for ZW organisms) specific promoters.

9. The method according to claim 1 wherein the step of creating transgenic animals includes the steps of targeting the transgene onto the sex chromosome of ES cells using the standard homologous recombination (gene-targeting) method, microinjecting the positive ES cells into recipient embryos to make chimeric mice, and breeding the chimeric mice to pass the ES-derived germ cells to the next generation to obtain the desired transgenic animals.

10. The method according to claim 1 wherein the step of creating transgenic animals includes the steps of targeting the transgene onto the sex chromosome of suitable nuclear donor cells by using the standard homologous recombination (gene-targeting) method, transferring the nuclei of the donor cells into enucleated oocytes by cell fusion or nuclear transfer, and activating the reconstituted oocytes and transfer them into pseudopregnant foster mothers to allow the embryos to develop into individuals with the transgene inserted onto the desired chromosome.

11. The method according to claim 1 wherein the step of creating transgenic animals includes the steps of co-transfecting the HSV-tk transgene with a selection marker gene into ES or nuclear donor cells by transfection, picking and growing neomycin-resistant clones, examining the transgene integration site by florescence in situ hybridization (FISH), expanding the clones with the transgene inserted onto the desired sex chromosome, and injecting the ES cells into embryos to make chimeric animals.

12. The method according to claim 1 wherein the step of creating transgenic animals includes the steps of creating transgenic animals from techniques selected from the group consisting of: pronuclear microinjection, retroviral vector transfection, lipofection, and sperm incubation, and examining the transgene integration site by FISH for each transgenic founder to search for individuals with the transgene inserted onto the desired sex chromosome.
